(19) United States
(12) Patent Application Publication
(10) Pub. No.: US 2004/0215158 A1
(43) Pub. Date: Oct. 28, 2004

(54) In line hose connector

(75) Inventor: Verne M. Anderson, Chicago, IL (US)

Correspondence Address: SCHIFF HARDIN, LLP, PATENT DEPARTMENT, 6600 SEARS TOWER, CHICAGO, IL, 60606-6473, US

(73) Assignee: Medpoint Corporation

(21) Appl. No.: 10/422,312

(22) Filed: Apr. 24, 2003

(51) Int. Cl. A61M 27/00

(52) U.S. Cl. 604/327; 604/544; 604/329; 604/540

(57) ABSTRACT

A connector for use in line in a catheter tube has a first part with a hose barb connected to the tube and a second part with a cylindrical opening and a circumferentially extending fastening strap. A hole is provided in the fastening strap. A gap is provided at the end of the fastening strap. A second part of the connector has a hose barb connected to another section of the catheter tube. The second part has a cylindrical portion that fits into the cylindrical opening of the first part. The second part also has a projection that may be positioned to enter the gap as the two pieces are fit together and which fits into the hole on the fastening strap after twisting of the pieces to latch the pieces together. The two pieces disconnect readily when a tension force is exerted that may otherwise cause injury to the wearer of the catheter.

### BACKGROUND OF THE INVENTION

1. Field of the Invention

The present invention relates generally to an in-line hose connector, and in particular to a releasable connector in a catheter tube.

2. Description of the Related Art

Catheters, in particular, urinary catheters are commonly used in medical situations. A urinary catheter is a soft plastic or rubber tube that is inserted into the bladder to drain the urine. The tube enters the body at the urethra and extends into the bladder. The other end of the tube commonly leads to a collection device, generally a bag in which urine is collected.

A type of catheter known as a Foley catheter or indwelling catheter has a small balloon at the end of the tube inside the bladder. The balloon remains un-inflated during insertion of the catheter and is inflated, such as by sterile water introduced through an inflation port tube, after insertion. The inflated balloon inside the bladder prevents withdrawal of the catheter tube. A typical balloon catheter holds about 5 to 10 ml of fluid. Removal of a Foley catheter is accomplished by draining the water from the balloon through the inflation port tube so that the balloon deflates and can be withdrawn through the urethra.

The catheter tube extends out of the body at the urethral opening and runs to the collecting bag. Ambulatory patients and other individuals may be able to wear a leg bag, a small collecting bag strapped to the leg. In this case, the catheter tube extends a short distance to the leg bag and may be taped to the leg. The wearer of the leg bag may find it inconvenient to disconnect the leg bag and connect to a bedside bag for sleep. However, for use in a reclining position, the collecting bag must be positioned to the side of the bed or gurney to ensure that the collecting bag is below the wearer's bladder. A larger collecting bag is used and is hung from the bed or gurney. This, however, exposes the catheter tube to being caught on objects and pulled.

Particularly in a hospital or emergency room setting, where there is often a flurry of activity, there is a high risk of the catheter tube being caught on an object being moved past the bed or gurney. Or even more serious is the chance that the catheter tube dangling from the side of the gurney catches on a door knob or other projection as the gurney is being moved about. Patients have occasionally been thrown to the floor from the gurney or even had the large diameter Foley balloon pulled through their urethra.

### SUMMARY OF THE INVENTION

The present invention provides a releasable connector for a tube, such as a catheter tube, which disconnects at the free will of the user or when excessive tension is exerted on the tube. In particular, the present tube connector has an in-line lumen of the same diameter as the tube, provides a fast and simple connect process and readily releases upon the application of tension force on the connector. In one embodiment, the present connector has a male portion having a tube connecting end that fits into one end of the tube in secure engagement and a second end having an end-most portion of a first diameter, and a second diameter portion of a larger diameter than the first diameter portion set back from the free end of the second end, and a small projection on the second diameter portion. The connector also has a female portion having a tube connection end that fits into an end of the tube in a secure engagement. The other end of the female portion has a receiving opening into which the first diameter end most portion of the male connector is received. The receiving end has a longitudinal gap and a circumferential fastening flap with a free end at the gap. The fastening flap has an opening or hole into which the projection on the male portion is received.

### BRIEF DESCRIPTION OF THE DRAWINGS

FIG. 1 is a perspective view of the present in-line connector with a collecting bag according to the present invention;

FIG. 2 is an enlarged perspective view of the in-line connector;

FIG. 3 is an exploded perspective view of the in-line connector in a disassembled state;

FIG. 4 is a partial cut-away perspective view of the connector in an assembled state;

FIG. 5 is a side cross section through the assembled connector;

FIG. 6 is an enlarged perspective view of the latching mechanism of the present connector; and

FIGS. 7, 8 and 9 are enlarged side views of embodiments of a latching projection according to different embodiments of the invention.

### DETAILED DESCRIPTION OF THE PREFERRED EMBODIMENTS

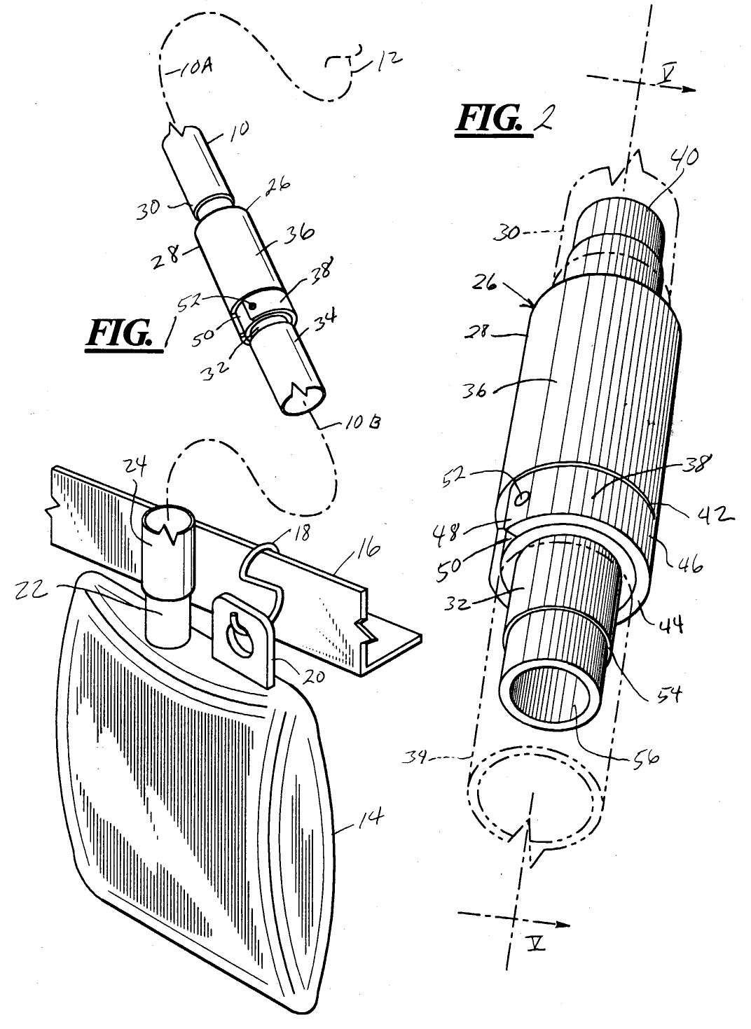

FIG. 1 shows a tube 10 for a urinary catheter. The tube 10 leads from a wearer's body at end 12 to a collecting bag 14. The end 12 at the wearer's body has been inserted into the urethra of the individual, preferably sufficiently far so that the free end is within the bladder of the wearer. The free end of the tube 10 may be plain or may be provided with a balloon or other device to assist in retaining the catheter in place, as is known.

Since the catheter tube 10 works primarily by gravity, it is important to keep the collecting bag below the bladder of the wearer. The wearer or patient is commonly reclining or seated, and may be unable to stand or walk, or at least restricted from rising and walking for the present. Thus, the need for the catheter to drain the bladder. The wearer is therefore on a bed, gurney, chair, wheelchair, or the like, and the collecting bag 14 is often supported on the bed, gurney, etc. For example, a frame member 16 of a bed or wheelchair is shown in FIG. 1. Hooked onto the frame member 16 is a support hook 18 that in turn is connected to a support tab 20 of the bag 14. The bag 14 includes an inlet 22 having a hose connector to which an end 24 of the tube 10 is connected.

In the prior art, the tube 10 is in one piece. However, according to the present invention the catheter tube 10 is in two pieces, an upper piece 10A and a lower piece 10B, such as by being cut or by providing two separate pieces of tubing, and a connector 26 is provided in-line with the tube 10. The connector 26 is provided at a position along the length of the tube 10, preferably at a point closer to the wearer's body than to the drain bag 14. The connector is preferably worn near the inside of the thigh. The connector 26 has a first part 28 connected to an end 30 of the upper tube piece 10A and a second part 32 connected to an end 34 of the lower tube piece 10B. The first part 28 includes a main body portion 36 and a fastening strap 38.

Referring now to FIG. 2, the connector 26 has the first part 28 and the second part 32 connected together. The first part 28 has a hose fitting or hose barb 40 over which the end 30 of the upper tube 10A is placed. The hose barb 40 may be smooth, rough, have a toothed surface or, as shown, have a stepped surface of increasing diameter. The stepped surface may have one step, two steps, three steps or more. The hose end 30 is pushed onto the hose barb 40 at least over the two illustrated steps, and may be pressed against the main body portion 36.

The main body portion 36 of the first part 28 is cylindrical and of a larger outside diameter than the hose 10. The outer surface is preferably smooth to avoid irritating the wearer's skin, although ridges or other grip enhancing surface textures may be provided. The end of the main body portion 36 opposite the hose barb 40 has the fastening strap 38, which has a cylindrical surface aligned with the surface of the main body portion 36 and is separated therefrom by a slot 42. The slot 42 cuts the fastening strap 38 partially free of the main body portion 36. The slot 42 extends about 180° around the cylindrical main body 36. The part of the main body 36 opposite the fastening strap 38 extends to an end 44 of the main body portion 36. The fastening strap 38 is of a width to extend from the slot 42 to the end 44.

The fastening strap 36 extends part way about the circumference of the cylindrical main body portion 36 and has a fixed end 46 and a free end 48. The free end 48 is spaced by an acceptance gap 50 from the part of the main body 36 that lies as a continuation of the circumferential extent of the fastening strap 38. The free end 48 has an opening or hole 52.

The second part 32 of the connector 26 has a hose connector or hose barb 54 over which the end 34 of the hose part 10B is fit. The balance of the second part 32 fits into the first part 28 when in a fastened position. The lumen or opening 56 through the second part 32 is seen. The lumen 56 continues through the first part 28 without interruption. The lumen 56 is preferably the same diameter of the lumen of the tube 10 and is smooth and aligned with the lumen of the tube 10 so as to present little or no resistance to liquid flow.

Turning now to FIG. 3, the connector 26 is disassembled. The first part 28 has the hose barb 40, the main body part 36 and the fastening strap 38 separated from the main body part 36 by the slot 42. The hole 52 is near the free end 48 of the fastening strap 38. The hole 52 preferably passes all the way through the strap 38 to provide a visual clue to the user for connection of the device. However, the hole may be only a recess in the underside of the strap into which the projection fits. The acceptance gap 50 between the free end 48 of the fastening strap 38 and the end of the main body portion 36 is better seen in this view. The acceptance gap 50 is of a width sufficient to provided easy connection of the first part 28 and the second part 32 together, as will be discussed in greater detail hereinafter. In one embodiment, the slot 42 extends about 180° of the circumference of the cylindrical main body portion 36, and the hole 52 is provided at about the 90° position. In other words, the hole 52 is set at a right angle to the ends of the slot 42. If the strap 38 is to be made more flexible, it is foreseen to make the strap longer and place the hole 52 at about 120° from the fixed end of the strap. It is of course possible within the scope of this invention to make the slot longer or shorter and to move the hole position to different places.

Inside the first part 28 beginning from its open end is a first diameter opening 58 and a second smaller diameter opening 60 defining an annular surface 62. The first diameter opening 58 lies under the fastening strap 38, and the second diameter opening 60 lies within the main body portion 36. The openings 58 and 60 are both cylindrical and concentric.

The second part 32 has the hose barb 54 at its end, followed by a larger diameter portion 64 and a smaller diameter portion 66. Both portions 64 and 66 are cylindrical in shape. The smaller diameter portion 66 is sized to fit into the second smaller diameter opening 60 of the first part and the larger diameter portion 64 is sized to fit into the first larger diameter opening 58 of the first part. Preferably, these parts are sized for a close fit but one that is not too tight, since only a small amount of friction should exist to permit ready movement of the two connector parts 28 and 32 relative to one another.

The second part 32 has an annular surface 68 between the larger diameter portion 64 and the smaller diameter portion 66.

To provide a liquid tight seal between the two parts 28 and 32, an o-ring 70 is provided in a channel 72 formed in the smaller diameter portion 66. The o-ring 70 is of dimensions to contact the inside surface of the smaller diameter opening 60 of the first part 28 so that the o-ring 70 is in compression between the surface of the smaller diameter opening 60 and the channel 72. A liquid tight seal is ensured.

The larger diameter portion 64 of the second part 32 has on its outer surface a projection 74. The projection 74 is of a size and is positioned on the larger diameter portion 64 at a position to fit into the hole 52 of the fastening strap 38.

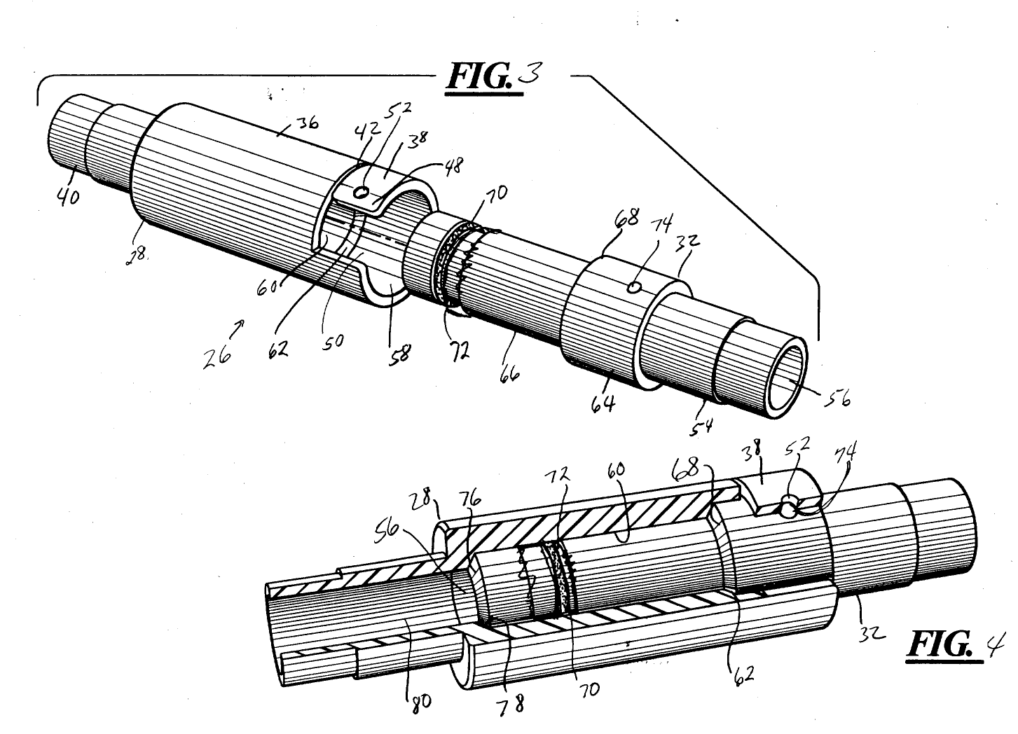

With reference to FIG. 4, the two parts 28 and 32 are shown connected together. The projection 74 is within the hole 52 of the fastening strap 38, thereby providing resistance to axial movement of the two parts 28 and 32 relative to one another. The projection 74 is released by the fastening strap 38 by the outward flexure of the fastening strap 38 upon the application of sufficient force to pull the pieces apart along the axial direction. For example, a pulling force on the tube 10 will cause the connector 26 to release.

A further surface 76 is provided at the end of the smaller diameter portion 66 of the second part 32. A surface 78 is inside the first portion 28. The lumen 56 inside the second part 32 and a lumen 80 inside the first part 28 form a continuous passageway for the fluid flow, preferably with no change in diameter and no roughening or surface discontinuities to hinder the free flow of the fluid.

The o-ring 70 in the channel 72 is seen pressing against the inside surface of the smaller diameter opening 60 of the first part 28. The surface of the smaller diameter opening 60 is preferably smooth to limit wear of the o-ring 70 as the parts 28 and 32 are connected and disconnected and provide an uninterrupted sealing engagement.

In FIG. 5, the lumen 56 and 80 through the connector 26 is continuous, smooth and uninterrupted. The end 30 of the tube 10A is pressed onto the hose barb 40 and may be left with a gap between the end 30 of the tube 10A and the main body portion 36 as shown, or may be pressed up against the main body portion 36 so that no or very little gap is present. It is preferred that the tube 10A be pressed on with no gap to avoid catching on body hairs of the wearer.

Similarly, the tube section 10B is pressed onto the hose barb 54 either to leave a gap or to abut against the main body part 36 of the first part 28 and the larger diameter portion 64 of the second part 32. Preferably, no gap is present to avoid catching body hairs. The external diameter of the main body portion 36 is not much greater than that of the tube 10. It is optional to form the main body portion of the same outside diameter as the tube 10 provided sufficiently strong material is utilized for the connector 26. It is also possible, although not preferable, to make the main body portion 36 of a significantly larger diameter than the tube 10. This might aid in use of the device by a person who's fine motor skills are impaired.

The slot 42 which separates the fastening strap 38 from the main body portion 36 is shown spaced from the first pair of seating surfaces 62 and 68 by a short axial distance. The slot 42 may be positioned at about the same axial location as the seating surfaces 62 and 68 in other embodiments. Likewise, the o-ring 70 is near the end seating surfaces 76 and 78, but may be located farther away from the end seating surfaces 76 and 78. It is also contemplated to provide the o-ring 70 at or near to the seating surfaces 62 and 68, and maybe even abutting the seating surface 62 of the first part 28. It is also contemplated to shorten the axial extend of the connector 26 over that shown in FIG. 5, such as by shortening the smaller diameter portion 66 of the second part 32 and correspondingly shortening the smaller diameter opening 60 in the first part 28. Other changes in axial length may be made as well. It is further contemplated to lengthen the connector, such as by making various elements longer.

Various other sealing devices may be used. For example, the o-ring may be replaced by a gasket or packing at the surfaces 62 and 68 or a seal with a deformable lip.

FIG. 6 illustrates the technique for fastening the two parts 28 and 32 to one another. The second part 32 is inserted into the opening in the first part 28. The projection 74 is positioned so that it enters the first part 28 at the gap 50. When the second part 32 is fully inserted into the first part 28, the projection 74 is aligned with the hole 52. In the FIG. 6, the projection is in the position A. Rotating or twisting the second part 32 relative to the first part 28 moves the projection 74 first against the free end 48 of the fastening strap 38, then lifts the fastening strap 38 as the projection slides under the fastening strap 38, as shown at position B. Once the parts are twisted relative to one another sufficiently, the projection 74 reaches the hole 52 and the fastening strap 38 snaps down into place against the larger diameter portion of the second part 32. The projection 74 is held captive in the hole 52 so that the connector parts 28 and 32 are secured together.

In the view of FIG. 6, the second part 32 is twisted in a clockwise direction to move the projection 74 from the gap 50 to the hole 52. To release the parts of the connector 26, the second part 32 may be twisted further in the clockwise direction, again relative to the illustration of FIG. 6. This presses the projection 74 against the inside wall of the hole 52 and causes the fastening strap 38 to again lift, as shown. The projection 74 is released from the hole 52 and the second part 32 and first part 28 may be pulled apart in the axial direction. The fastening strap 38 of course snaps back down as soon as the projection 74 is out from under it.

The connected parts 28 and 32 may be released from one another by simply pulling in an axial direction. This results in the fastening strap 38 being lifted to release the projection from the hole 52 and the projection passing under the fastening strap 38 in the axial direction. The release of the connector by pulling replicates the release of the connector when undue stress is placed on the catheter tube 10. The release force in the pulling direction is of such magnitude that the connector does not release too easily, but does release possibly and preferably before tissue damage occurs for the wearer of the catheter.

The reconnection of the two parts 28 and 32 to one another is easily accomplished by anyone. The shapes of the part suggest the connection technique, so that it is apparent to someone seeing this for the first time that the second part 32 may be inserted into the first part 28 with the projection 74 in the gap 50 and then engagement may be accomplished by twisting.

It is noted that the projection 74 is high enough that it will generally not be possible to force it under the end 58 of the main body part that lies opposite the fastening strap 38. It is also generally not possible to twist the two parts 28 and 32 relative to one another much beyond the point at which the projection 74 lifts the fastening strap 38 since the fixed end 46 of the fastening strap 38 flexes upwardly by only a very small amount. It is further interesting to note that once the projection 74 is in the hole 52, twisting of the second part 32 in a counter-clockwise direction is difficult if not impossible, since the fastening strap 38 tends to pull down with greater force on the area surrounding the projection 74 during such twisting and hinders release of the connector by twisting in the opposite direction to the twist direction used to fasten the parts together.

The free end 48 of the fastening strap 38 may be chamfered, for example at its inside edge adjacent to the greater diameter portion 64, to ease lifting of the strap 38 and movement of the projection 74 thereunder. The interior of the hole 52 may also be chamfered, for example at the bottom edge, to ease lifting of the strap 38 for release of the connector.

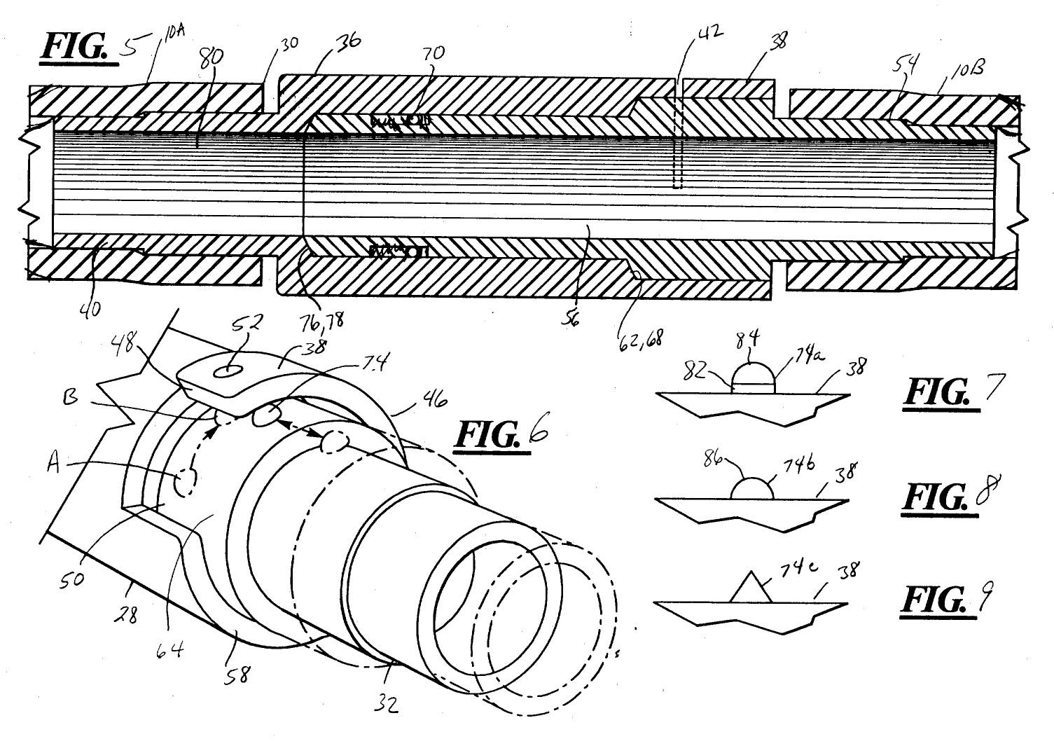

The shape of the projection 74 plays a roll in the ease of release and engagement of the present connector. For example, in FIG. 7, the projection 74 has a shape 74a, which defines a short cylindrical portion 82 extending perpendicularly from the fastening strap 38 and atop the cylindrical portion 82 is a hemispherical portion 84. The cylindrical portion of one embodiment has a length of 10/1,000 inch. In another embodiment as shown in FIG. 8, the projection 74b has only a hemispherical portion 86 on the fastening strap 38. A further alternative is shown in FIG. 9. The projection 74c has a conical shape. Each of these shapes of the projection 74 aid the fastening strap 38 in lifting to permit the present connector to engage and then to lift the fastening strap 38 to disengage.

The projection may have a different shape than those shown here. Likewise, the fastening strap may have a different configuration than that shown, and may include beveling or other shaping of the edges to change the engage and release characteristics of the present connector. The material of which the present connector is made, in particular, the hardness of the material, effects the engage and release characteristics of the connector, for example, by changing the flexibility of the fastening strap. The force required for engagement and disengagement may also be varied by varying the thickness of the fastening strap. In one embodiment, the connector is formed of a polymeric material, such as polycarbonate.

All external surfaces of the present connector preferably have radiused and chamfered edges to avoid scratching and irritation to the wearer. Since the device is typically worn against the inside of the thigh, preventing irritation is important. Preferably, knurled surfaces and raised collars are avoided. Although the illustrated connector is cylindrical in overall shape, it is possible that the outside surface of the connector or some external elements thereof may be square, rectangular or some other shape in axial cross section, such as to facilitate gripping by individuals with reduced dexterity.

The present connector works equally well regardless of which of the two parts is connected to the upstream end of the catheter tube.

Although other modifications and changes may be suggested by those skilled in the art, it is the intention of the inventor to embody within the patent warranted hereon all changes and modifications as reasonably and properly come within the scope of his contribution to the art.

1. A releasable connector for a catheter tube, comprising:
a first part having:
a hose connector for connection to a first part of the catheter tube;
a main body part connected to said hose connector and defining an opening;
a fastening strap connected to said main body part and defining a hole therein;
a second part for connection to said first part, said second part having:
a hose connector for connection to a second part of the catheter tube;
a portion for inserting into said opening in said main body part, said portion being connected to said hose connector; and
a projection on said second part that selectively fits into said hole in said fastening strap when said second part is in engaged connection to said first part.

2. A releasable connector as claimed in claim 1, wherein said fastening strap extends substantially circumferentially from a fixed end connected to said main body portion to a free end.

3. A releasable connector as claimed in claim 1, wherein said opening in said main body part is substantially cylindrical, and
said portion of said second part is substantially cylindrical to permit said first and second parts to be twisted relative to one another when said portion of said second part is in said opening of said second part.

4. A releasable connector as claimed in claim 1, wherein said main body portion of said first part defines a gap adjacent to said fastening strap, said gap being of a dimension to accept said projection on said second part during insertion of said portion of said second part into said opening of said first part.

5. A releasable connector as claimed in claim 1,further comprising:
an o-ring on said portion of said second part for sealing engagement with a surface of said opening of said first part when said second part is in engaged connection to said first part.

6. A releasable connector as claimed in claim 1, wherein said hole in said fastening strap passes entirely through said fastening strap.

7. A releasable connector as claimed in claim 1, wherein said opening in said main body part has a first diameter opening part and a second diameter opening part, and
said portion of said second part has a first diameter part and a second diameter part for cooperative engagement in respective ones of said first and second diameter opening parts of said first part when said second part is in engaged connection to said first part.

8. A releasable connector as claimed in claim 1, wherein said first and second parts define a lumen entirely through said connector, said lumen being substantially coaxial of a lumen of said tube when said second part is in engaged connection to said first part.

9. A releasable connector for a catheter tube, comprising:
a first part having:
a hose barb with an outer hose engaging surface and a lumen extending in an axial direction;
a main body portion having a substantially cylindrical exterior shape coaxial with said hose barb, said main body portion being connected to said hose barb, said main body portion defining an opening coaxial with said hose barb, said opening having a smaller diameter cylindrical portion and a larger diameter cylindrical portion;
a seating surface having an annular shape and being between said smaller diameter cylindrical portion and said larger diameter cylindrical portion of said opening;
a fastening strap connected at a fixed end to said main body portion, said fastening strap having a free end disposed in a circumferential direction from said fixed end, said fastening strap defining a hole adjacent said free end;
a second part for connection to said first part in an engaged condition, said second part including:
a hose barb having an outer hose engaging surface and a lumen extending in an axial direction;
a cylindrical insert portion for insertion into said opening of said first part when said first and second parts are connected in an engaged condition, said cylindrical insert portion having a first diameter insert part and a second diameter insert part on an outer surface thereof, said cylindrical insert part having a lumen coaxial with said lumen of said hose barb;
an annular seating surface between said first diameter insert part and said second diameter insert part, said annular seating surface of said second part abutting said annular seating surface of said first part when said first and second parts are connected in an engaged condition; and
a projection on said second part, said projection fitting into said hole of said fastening strap when said first and second parts are connected in an engaged condition.

10. A releasable connector as claimed in claim 9, wherein said main body portion defines a gap adjacent said free end of said fastening strap in a circumferential direction from said free end of said fastening strap;
said projection being accepted into said gap during insertion of said cylindrical insertion portion of said second part into said opening of said first part, said first part and said second part being rotatable about said axis to bring said projection into said hole in said fastening strap.

11. A releasable connector as claimed in claim 10, further comprising:
a channel formed on said cylindrical insertion portion of said second part; and
an o-ring in said channel for engagement to an interior surface of said opening in said main body portion.

12. A releasable connector as claimed in claim 10, wherein said projection has a substantially hemispherical surface.

13. A releasable connector as claimed in claim 10, wherein said projection has a substantially conical shape.

14. A releasable connector as claimed in claim 10, wherein said first part and said second part release from one another when subjected to a predetermined tension.

15. A catheter tube, comprising:
a first tube portion for insertion into an orifice of a wearer;
a second tube portion for connection to a drain;
a releasable connector including a first part and a second part connected between said first and second tube portions;
said first part having:
a hose barb with an outer hose engaging surface and a lumen extending in an axial direction;
a main body portion having a substantially cylindrical exterior shape coaxial with said hose barb, said main body portion being connected to said hose barb, said main body portion defining an opening coaxial with said hose barb, said opening having a smaller diameter cylindrical portion and a larger diameter cylindrical portion;
a seating surface having an annular shape and being between said smaller diameter cylindrical portion and said larger diameter cylindrical portion of said opening;
a fastening strap connected at a fixed end to said main body portion, said fastening strap having a free end disposed in a circumferential direction from said fixed end, said fastening strap defining a hole adjacent said free end;
said second part for connection to said first part in an engaged condition, said second part including:
a hose barb having an outer hose engaging surface and a lumen extending in an axial direction;
a cylindrical insert portion for insertion into said opening of said first part when said first and second parts are connected in an engaged condition, said cylindrical insert portion having a first diameter insert part and a second diameter insert part on an outer surface thereof, said cylindrical insert part having a lumen coaxial with said lumen of said hose barb;
an annular seating surface between said first diameter insert part and said second diameter insert part, said annular seating surface of said second part abutting said annular seating surface of said first part when said first and second parts are connected in an engaged condition; and
a projection on said second part, said projection fitting into said hole of said fastening strap when said first and second parts are connected in an engaged condition.
